(19) United States
(12) Patent Application Publication
(10) Pub. No.: US 2016/0256647 A1
(43) Pub. Date: Sep. 8, 2016

(54) PROTECTIVE COVER FOR A TRACH TIE

(71) Applicant: Felita Yvette THOMAS, Cincinnati, OH (US)

(72) Inventor: Felita Yvette THOMAS, Cincinnati, OH (US)

(21) Appl. No.: 14/638,181

(22) Filed: Mar. 4, 2015

(51) Int. Cl. A61M 16/04

(52) U.S. Cl. CPC A61M 16/047; A61M 16/0497; A61M 2240/00

(57) ABSTRACT

A protective cover for use with a tracheostomy tube inserted into a patient's trachea comprises a middle portion including an opening for a tracheostomy tube to pass through, a first strap portion extending from the middle portion in a first direction, and a second strap portion extending from the middle portion in a second direction opposite to the first strap portion. The cover's middle portion and strap portions cover and substantially prevent secretions from reaching underlying equipment such as a trach tie and a neck plate used to secure the tracheostomy tube to the patient, while allowing the tracheostomy tube to be used for breathing.

### FIELD OF THE INVENTION

The present invention relates in general to respiratory devices, and in particular to a protective cover for a tracheostomy tie that protects the patient's skin beneath the tracheostomy tie and the external surfaces of the tracheostomy tube.

### BACKGROUND OF THE INVENTION

There are many patients in the U.S. who, for various reasons, require a tracheostomy tube in order to maintain their airway. A tracheostomy tube is a short curved tube inserted through a surgical hole in the patient's neck. The tracheostomy tube typically has an external flange held in place with a tracheostomy tie (“trach tie”) threaded through an opening in the flange and turned back upon itself so that it may be securely fastened in place around the neck.

Patients who require a tracheostomy tube and tie, and in particular hospital-bound tracheostomy patients, must continuously manage the problem of mucus and other bodily secretions exiting the tube and irritating the skin underneath their trach tie. Further, harmful secretions often find their way between the patient's tender neck skin and the external surfaces of the tracheostomy tube, such as the external flange of the tracheostomy tube, potentially causing irritation and infection of the underlying skin.

The prolonged need for a tracheostomy tube therefore brings about the possibility of prolonged exposure of the patient's skin to harmful secretions, often leading to breakdown of the skin beneath the trach tie, as well as the skin beneath the external surface of the tracheostomy tube flange. This skin breakdown is particularly troublesome in infants and pediatric patients, due to this patient population typically having soft, tender neck skin.

In light of the above, it would be desirable to provide an apparatus and method for substantially preventing harmful bodily secretions from making continual intimate contact with the skin beneath a patient's trach tie and beneath the hard plastic external flange or neck plate of the tracheostomy tube. There is also a need for a protective cover for trach ties that protects and substantially prevents skin breakdown while still allowing the tracheostomy tube to be used for breathing.

### SUMMARY OF THE INVENTION

The present invention provides a protective cover for substantially preventing harmful bodily secretions from making continual intimate contact with the skin beneath a patient's trach tie and beneath the hard plastic neck plate of the tracheostomy tube.

In one aspect, the invention provides a protective cover for use with a tracheostomy tube inserted into a patient's trachea, the cover comprising a middle portion including an opening for a tracheostomy tube to pass through; a first strap portion extending from the middle portion in a first direction; and a second strap portion extending from the middle portion in a second direction opposite to the first strap portion, wherein the cover and substantially prevents secretions exiting the tracheostomy tube from reaching underlying equipment used to secure the tracheostomy tube to the patient, while allowing the tracheostomy tube to be used for breathing.

The protective cover can be in the form of a “bow-tie” in which the width (i.e. the length measured from top to bottom) of the first and second strap portions and the middle portion are the same (typically about 2 inches wide), or the protective cover can be in the shape of a “bib” in which the middle portion extends further downward from the lower end of the strap portions a few inches. Each of the strap portions can include a Velcro strip for securing the strap portions at the back of the patient's neck. Further, a curtain can be secured to the back surface of the cover in order to catch secretions passing back through the opening.

The nature and advantages of the present invention will be more fully appreciated from the following drawings, detailed description and claims.

### BRIEF DESCRIPTION OF THE DRAWINGS

The aspects and advantages of the invention will be better understood from the following detailed description, with reference to the accompanying drawings, in which:

FIG. 1 is a frontal view of a patient with a stoma as a result of a tracheostomy;

FIG. 2 is a frontal view of the patient of FIG. 1 with a trach tube and tie combination for use with the stoma;

FIG. 3A is a frontal view of the patient of FIG. 2 wearing a “bow-tie” embodiment of the protective cover of the invention;

FIG. 3B is a front, plan view of the “bow-tie” embodiment of the protective cover of FIG. 3A;

FIG. 4A is a frontal view of the patient of FIG. 2 wearing a “bib” embodiment of the protective cover of the invention;

FIGS. 4B and 4C are front and back plan views, respectively, of the “bib” embodiment of the protective cover of FIG. 4A;

FIG. 5 is a lateral perspective view of the “bib” embodiment secured about the neck of a patient and covering a trach tube and tie combination.

### DETAILED DESCRIPTION OF THE INVENTION

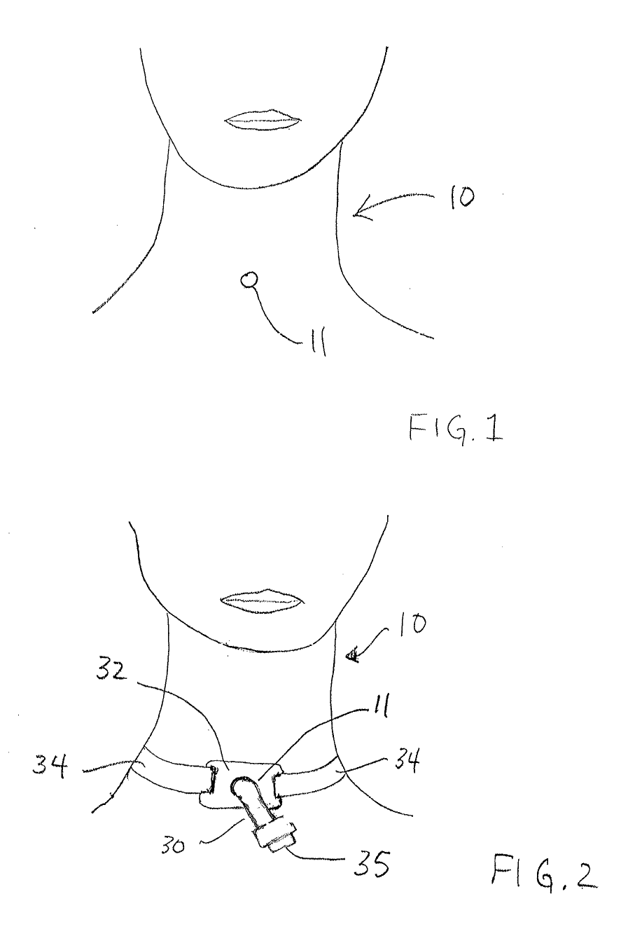

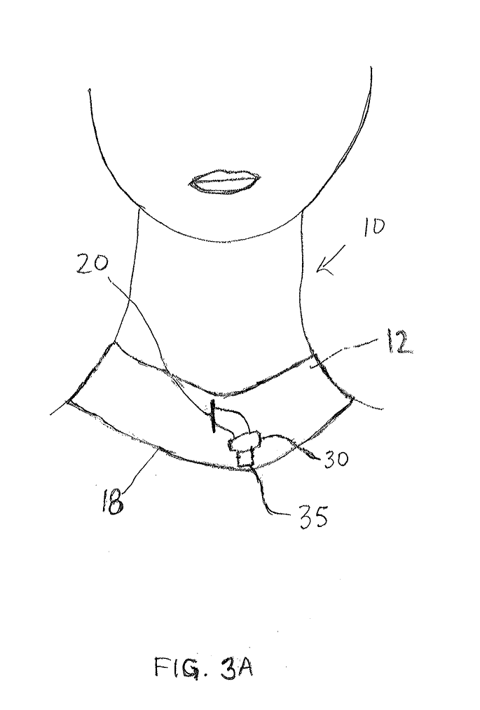

Referring to FIG. 1, a patient 10 is illustrated with a stoma or surgical orifice 11 at the anterior, lower neck. The surgical orifice 11 is the result of a surgical tracheostomy procedure, such as from a laryngectomy. The result of this surgical procedure is that the patient 10 has a permanent connection of the trachea or windpipe to the outside. It is through this surgical orifice 11 that respiration takes place, typically via either an endotracheal tube or a tracheostomy tube, as shown in FIG. 2.

FIG. 2 illustrates a typical set up for a tracheostomy tube and tie combination. The tracheostomy tube 30 shown is representative of a variety of different tracheostomy tubes available in the art. The tube 30 is typically made of a PVC material and inserted through the surgical orifice 11. The tube 30 is positioned over its exit from the surgical orifice 11 by a thin, flexible and fixed neck plate or flange 32 adapted to enable some form of band or tracheostomy tie 34 (“trach tie”) to be attached, which is tied or otherwise secured around the neck of the patient 10 to support the tube 30 in place. The neck plate 32, when tied by the trach tie 34, serves to stabilize the tube 30 over the surgical orifice 11.

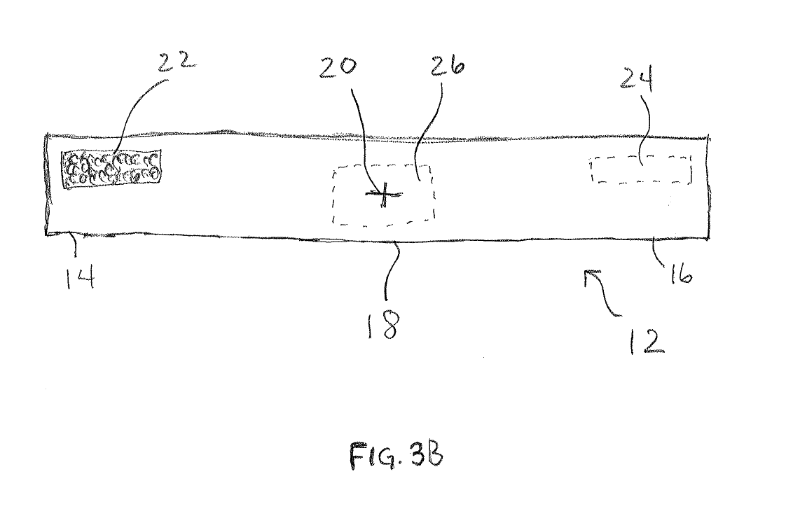

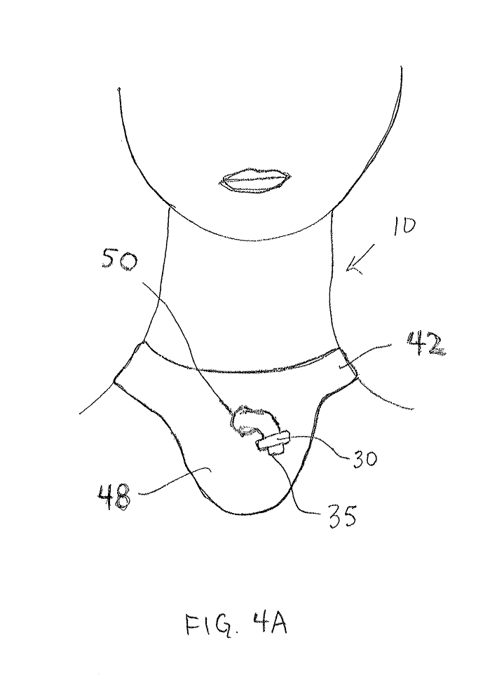

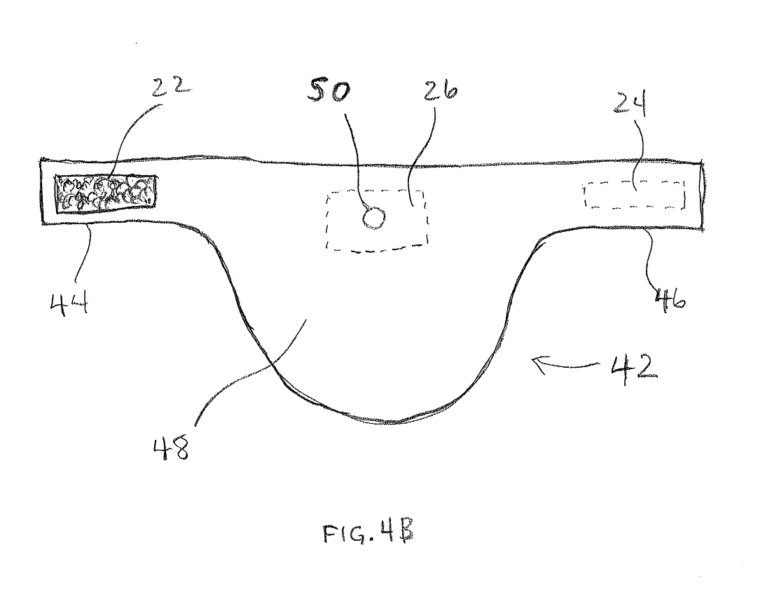

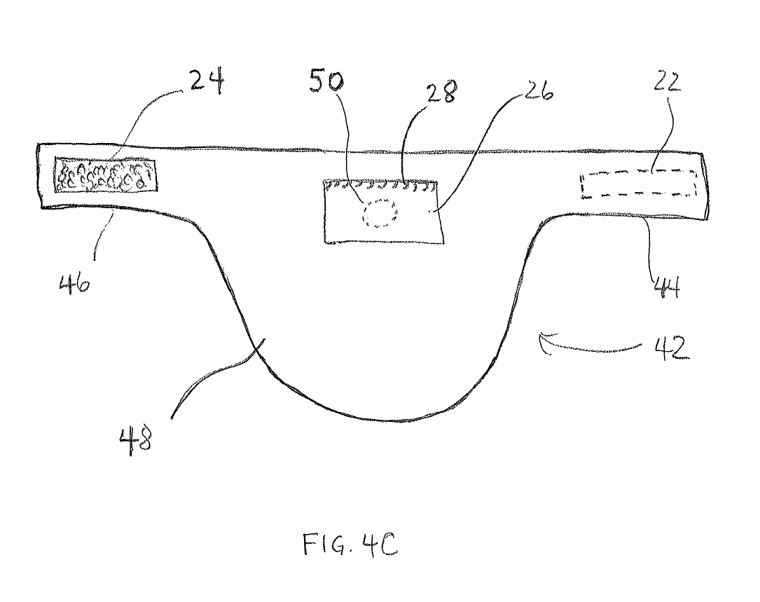

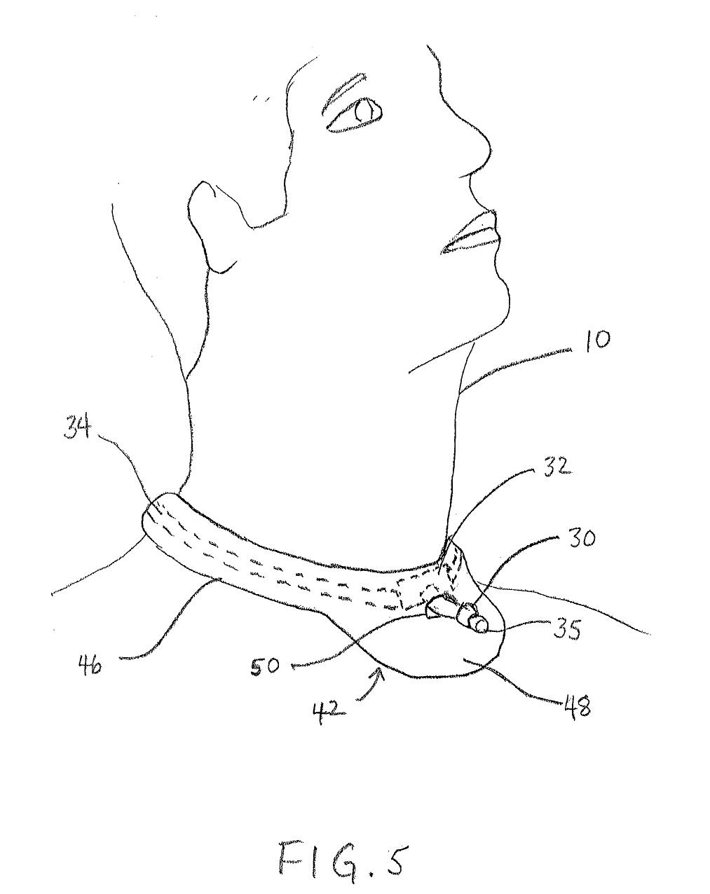

As shown in FIGS. 3A and 3B, one embodiment of a protective cover of the present invention is a “bow-tie” embodiment 12, which is typically preferred by adults. A “bib” embodiment 42, which can be made for any patient but is typically preferred for children, is shown in FIGS. 4A-4C and FIG. 5. Either embodiment 12, 42 can be used to prevent mucous and other bodily fluids exiting a tracheostomy tube from reaching and soiling the underlying trach tube plate and tie combination as well as from soiling shirts and other clothing items. In the bow-tie embodiment of FIGS. 3A and 3B, the width (i.e. the length measured from top to bottom) of the strap portions 14, 16 and the middle portion 18 of the cover 12 are the same. Typically this width is between one and two inches, but can range from one (1) inch up to four (4) inches in width. In the “bib” embodiment shown in FIGS. 4A-4C and FIG. 5 the width measured from top to bottom of the middle portion 48 of the cover 42 is larger than the strap portions 44, 46. The middle portion 48 can extend further downward from the lateral strap portions 44, 46, for example, less than an inch and up to four to six inches (or more, if needed) further down (i.e. from the bottom ends of the straps when worn), depending on patient preference and tracheostomy needs (i.e. amount of secretions exiting the tube 30). As a non-limiting example, the strap portions can be 2 inches wide and the middle portion can be from less than one inch up to about six inches wider than the strap portions, measured from top to bottom when worn. The transition in width from the middle portion to the strap portions can be gradual, as illustrated, with the lateral-most portions of the straps which include the Velcro strips 22, 24 having the smallest width. In either embodiment, the width of the strap portions and the middle portion should be enough to cover and thus substantially protect the underlying trach tie 34 and flange 32 from secretions exiting the end 35 of the trach tube 30.

As illustrated in FIGS. 3A and 3B, an opening 20 of the bow-tie protective cover embodiment 12 is intended to receive the trach tube 30 therethrough, with the middle portion 18 and hole 20 centered over the middle of the neck plate 32, which is centered over the surgical orifice 11 (see FIGS. 1 and 2) and the strap portions 14, 16 secured by Velcro straps 22, 24 behind the neck. Applied in this manner, the cover 12 can prevent harmful bodily fluids from wetting/soaking the trach tie 34 or the neck plate 32 (see e.g. FIG. 5), the patient's neck skin underneath, or any other clothing items nearby, thereby substantially preventing skin irritation and infection around the trach site. The external flange 32 and the trach tie 34 are sheltered by the protective cover 12 so that harmful secretions passing out of the end 35 of the trach tube 30 are prevented from reaching them.

The opening through the middle portion, shown in FIGS. 3A, 3B as a slit 20 and in FIGS. 4A-C and FIG. 5 as a small hole 50 allowing the trach tube 30 to pass therethrough, should be large enough to permit the trach tube to easily pass through without restriction, yet small enough to catch or prevent secretions from passing backwards through the slit 20 or hole 50 in a retrograde fashion. An optional piece of material in the form of a curtain 26 can be secured to the back surface of the cover in order to catch any secretions that may happen to pass back through the opening in a retrograde fashion. As illustrated in FIG. 4C, this curtain 26 can be secured by stitching 28 (see FIG. 4C) to the back surface of the cover. The opening (20, 50) of the inventive cover thus allows the tube to remain open to the air for breathing therethrough, or to be connected to a ventilator circuit component or other breathing device, while the cover protects the flange and trach tie from getting soiled with secretions.

Plan views showing the details of the construction of the preferred embodiments can be seen in FIGS. 3B, 4B, and 4C. FIGS. 3B and 4B show a first, front surface of the cover 12, 42, respectively, and FIG. 4C shows a second, back surface of the inventive cover 42. As can be seen in FIGS. 3B and 4B, the front surface of the cover includes two band-shaped strap portions 14 and 16, 44 and 46, each of which extend from opposing ends of the middle portion 18, 48 of the cover. As noted above, the width (i.e. the length measured from top to bottom) of the strap portions 14, 16 and the middle portion 18 of the “bow-tie” embodiment 12 of FIGS. 3A and 3B are the same, typically between one and two inches and up to four inches wide from top to bottom, while in the “bib” embodiment shown in FIGS. 4A-C the middle portion 48 of the cover 42 is wider and can extend further down from one to six inches. The middle portion 18, 48 of these embodiments has an opening 20, 50 for a tracheostomy tube 30 to pass through. The first strap portion 14, 44 extends from the middle portion 18, 48 in a first direction, and the second strap portion 16, 46 extends from the middle portion 18, 48 in a second direction, opposite the direction of the first strap portion.

In use, the strap portions 14, 16 of FIG. 3B (and 44, 46 of FIG. 4B) are placed around the neck of the patient 10, so that the middle portion 18, 48 is centered over the neck plate 32 and the opening 20, 50 is centered over the tracheostomy tube 30 as it passes out of the surgical orifice 11 (see FIG. 1) and through the neck plate 34 (see FIG. 2). Thus, the inventive cover, including the middle portion and the strap portions, can cover and substantially block secretions from reaching underlying equipment (i.e. the neck plate 32 and trach tie 34) used to secure the tracheostomy tube 30 to the patient, while still allowing the tracheostomy tube to be used for breathing. Specifically, the strap portions 14, 16 (and 44, 46) cover the underlying trach tie 34 and the middle portion 18, 48 covers the underlying neck plate 32. As noted above, a curtain 26 can be secured to the back surface of the cover in order to catch any secretions passing back through the opening 20, 50 (see, e.g., FIG. 4C).

As shown in FIGS. 3B, 4B and 4C, the strap portions 14, 16 and 44, 46 can be secured to one another at the back of the patient's neck in any manner known in the art. As a non-limiting example, each of the strap portions can include a Velcro-type fastener or strip 22, 24, respectively, for securing the strap portions at the back of the patient's neck. FIGS. 3B and 4B show a first, front surface and FIG. 4C shows a second, back surface of the inventive cover. It can be appreciated from viewing these figures that the Velcro strips 22, 24 are placed on opposite surfaces of the cover near the ends of the strap portions 14, 16 or 44, 46. In this manner, the ends of the strap portions can overlap and be secured to one another behind the neck via reversible connection of the Velcro strips 22, 24. The opening 20 in the middle portion 18 of the cover 12 is illustrated in FIG. 3B as a slit opening for the tracheostomy tube to pass through. However, the opening 20 can also be a small hole 50 as illustrated in FIGS. 4A-C and FIG. 5, or any other type of opening known in the art, so long as the opening allows the tracheostomy tube 30 to pass through without restriction, yet is not so large as to allow secretions coming from the end 35 of the tube 30 to freely flow back through the opening 20 towards the patient. An optional piece of material in the form of a curtain 26 can be secured to the back surface of the cover in order to catch any secretions that may pass back through the opening 20 in a retrograde fashion. As shown in FIG. 4C, the curtain 26 can be stitched 28 to the back surface of the cover at its top portion, and allowed to drape downward over the opening 50. The curtain 26 can include a slit 27 at its midline to allow the curtain 26 to be draped around the tube 30 as it exits the opening 50.

The protective cover of the invention is preferably constructed in various sizes to accommodate variation in size of the patient. For use with infants and children, small sizes having a length (i.e. a length measured between the lateral ends of the strap portions 14 and 16) adequate to accommodate a neck size of between about six inches to about eleven inches is desirable. For use with most adult females, the length of between about ten inches to about fifteen inches is desirable. Larger sizes, primarily for men or obese patients with large neck circumferences, should accommodate a neck size of between about fourteen inches to about twenty five inches. Thus, the cover of the invention can have a range of lengths measured between the lateral ends of the strap portions (14, 16 or 44, 46) of between six (6) inches and twenty-five (25) inches. The cover can be constructed of a variety of disposable or non-disposable and washable materials, such as for instance, cotton, polyester, blends of such materials, and a variety of other cellulosic or different water repellent or water resistant materials, as is known in the art.

While the present invention has been illustrated by the description of embodiments and examples thereof, it is not intended to restrict or in any way limit the scope of the appended claims to such detail. Additional advantages and modifications of the details of the invention will be readily apparent to those skilled in the art. Accordingly, departures may be made from such details without departing from the scope of the invention.

What is claimed is:

1. A protective cover for use with a tracheostomy tube inserted into a patient's trachea, the cover comprising:
a) a middle portion including an opening for a tracheostomy tube to pass through;
b) a first strap portion extending from the middle portion in a first direction; and
c) a second strap portion extending from the middle portion in a second direction opposite to the first strap portion, wherein the cover and substantially prevents secretions exiting the tracheostomy tube from reaching underlying equipment used to secure the tracheostomy tube to the patient, while allowing the tracheostomy tube to be used for breathing.

2. The protective cover of claim 1, the cover including a front surface and a back surface, the cover further comprising a curtain secured to the back surface of the cover in order to catch secretions passing back through the opening.

3. The protective cover of claim 2, wherein the curtain includes a slit at its midline to allow the curtain to be draped around the tracheostomy tube.

4. The protective cover of claim 2, wherein each of the strap portions includes a Velcro strip placed on opposite front and back surfaces of the cover near the ends of the strap portions for securing the cover around the patient's neck.

5. The protective cover of claim 1, wherein the length of the cover measured between the lateral ends of the strap portions is between six inches and twenty-five inches.

6. The protective cover of claim 1, wherein the width measured from top to bottom of the middle portion is the same as the width of the strap portions.

7. The protective cover of claim 6, wherein the width of the strap portions and the middle portions is between one inch and four inches.

8. The protective cover of claim 1, wherein the width measured from top to bottom of the middle portion is larger than the width of the strap portions.

9. The protective cover of claim 8, wherein the width of the strap portions is between one inch and four inches and the width of the middle portion is up to six inches larger than the strap portions.
